(19) United States
(12) Patent Application Publication
(10) Pub. No.: US 2014/0053843 A1
(43) Pub. Date: Feb. 27, 2014

(54) Mask for a Breathing Therapy Machine Having Improved Tilt Adjustment and Connection Mechanism

(71) Applicants: Allan R. Jones, JR., Derry, PA (US); Daniel Conn, II, Davidsville, PA (US)

(72) Inventors: Allan R. Jones, JR., Derry, PA (US); Daniel Conn, II, Davidsville, PA (US)

(73) Assignee: DEVILBISS HEALTHCARE LLC, Somerset, PA (US)

(21) Appl. No.: 13/595,101

(22) Filed: Aug. 27, 2012

(51) Int. Cl. A61M 16/06

(52) U.S. Cl. 128/205.25

(57) ABSTRACT

A design for a mask capable of use with a breathing therapy machine which allows rotation of the mask portion with respect to the remainder of the mask and provides a connection member for the connection to the hose of the breathing therapy machine which splits into two hoses and allows for the crimping of the connection without obstructing airflow.

### FIELD OF THE INVENTION

This invention is related to the field of breathing therapy machines, such as continuous positive airway pressure (CPAP) or bi-level positive airway pressure (Bi-PAP) machines of the type typically used to treat patients suffering from breathing disorders, such as hypopnea or apnea, and, in particular, is related to the user interface for such devices.

### BACKGROUND OF THE INVENTION

Breathing therapy machines are well known in the art for use in the treatment of a number of respiratory conditions, such as sleep apnea and hypopnea, by supplying a continuous positive pressure to a patient's airway while the patient sleeps. A typical CPAP apparatus has a user interface consisting of a mask which typically covers the user's nose, mouth or both, with the mask being constructed from a soft, pliable material or hard plastic with a soft, pliable gasket around the edges thereof capable of forming a seal between the mask and the user's face. The mask is typically kept in place with some form of headgear which straps around the user's head to keep the mask in contact with the user's face. In addition, the mask is typically connected to the breathing therapy machine via a hose.

One important characteristic of the user interface is comfort. As should be realized, many people have differing sleep habits and positions and will be reluctant to use a machine which requires a particular sleep position or which has impaired performance when the user changes position. Prior art masks suffer from a variety of problems, including disengagement of the mask from the user's face resulting in a large leakage component and crimping of the hoses, causing decreased airflow.

What is required to address these deficiencies is a mask which allows free rotation of the head, in both the up and down directions as well as from side to side, and an interface to the breathing therapy machine which reduces crimping and restricted airflow.

### SUMMARY OF THE INVENTION

The present invention solves the deficiencies inherent in prior art masks used with breathing therapy machines. In particular, the mask provides infinite rotational adjustment though a range of motion allowing for constant correction due to changes in forces and facial expressions. The mask is provided with a hose interface which allows the mask to rotate about an axis. Preferably, the hose will have dual hose connection points on opposite sides of the mask and a connection point with the headgear apparatus which is co-located with the hose connection points. The mask is thus able to rotate with respect to the headgear connections and independent of the hose connections, thereby allowing the mask itself to maintain a connective seal with the user's face, given a variety of sleeping position and movements which may be made by the user during sleep.

The mask has the advantages of improved comfort for the user as well as the ability to minimize leaks cause by the mask being pulled away from the face due to torque forces applied by the hose connection as the user moves during sleep.

### BRIEF DESCRIPTION OF THE DRAWINGS

FIG. 1 is a perspective drawing of the device of the present invention, showing all parts.

FIG. 2 is an exploded view of a portion of the device.

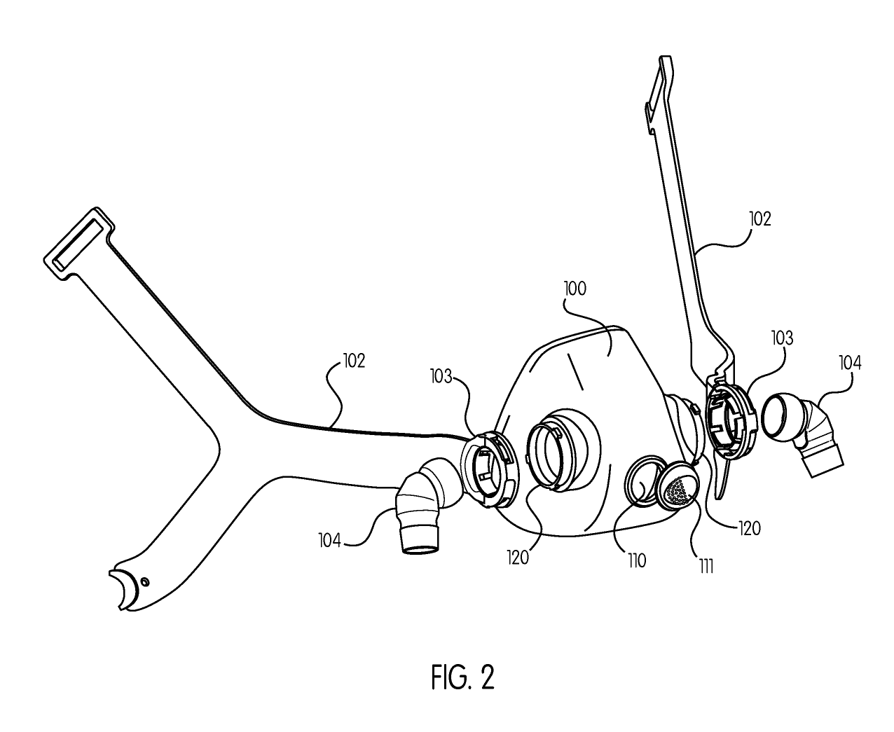

FIG. 3 shows the mask portion and its various parts.

FIG. 4 shows the headgear members of the device.

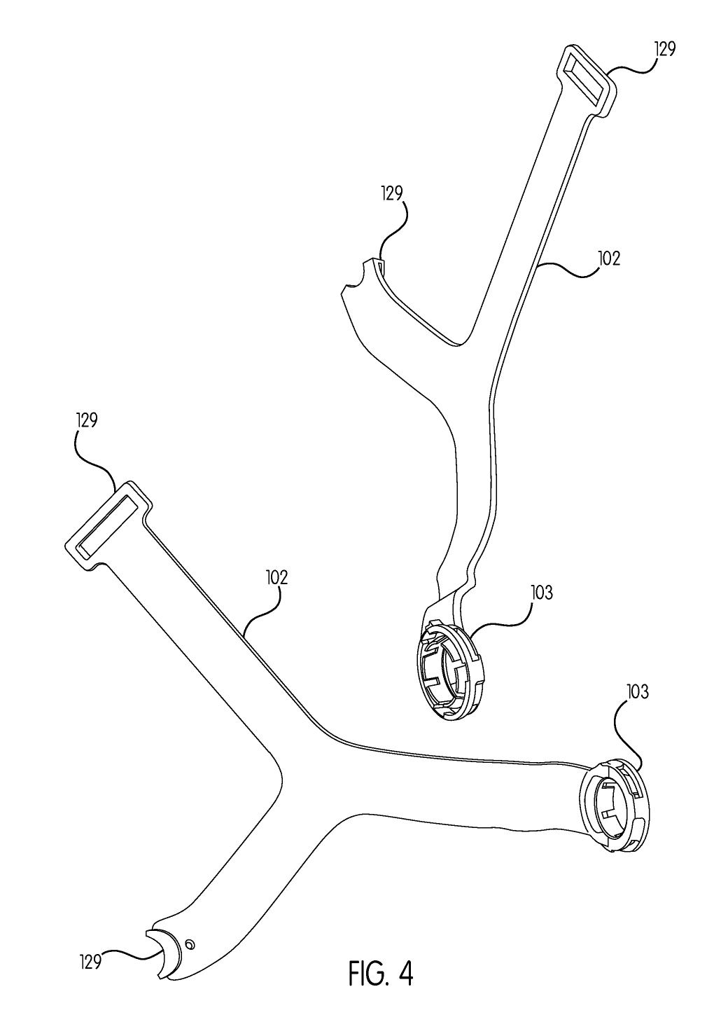

FIG. 5 shows the connection members for connecting the hoses of the device to the mask portion.

FIG. 6 shows the Y-connector portion for connecting the hoses of the device to a breathing therapy machine.

FIG. 7 shows a cross section of the Y-connector portion of FIG. 6.

FIG. 8 shows the mask of the present invention positioned on a human head.

### DETAILED DESCRIPTION OF THE INVENTION

The invention will now be described in detail with reference to the figures.

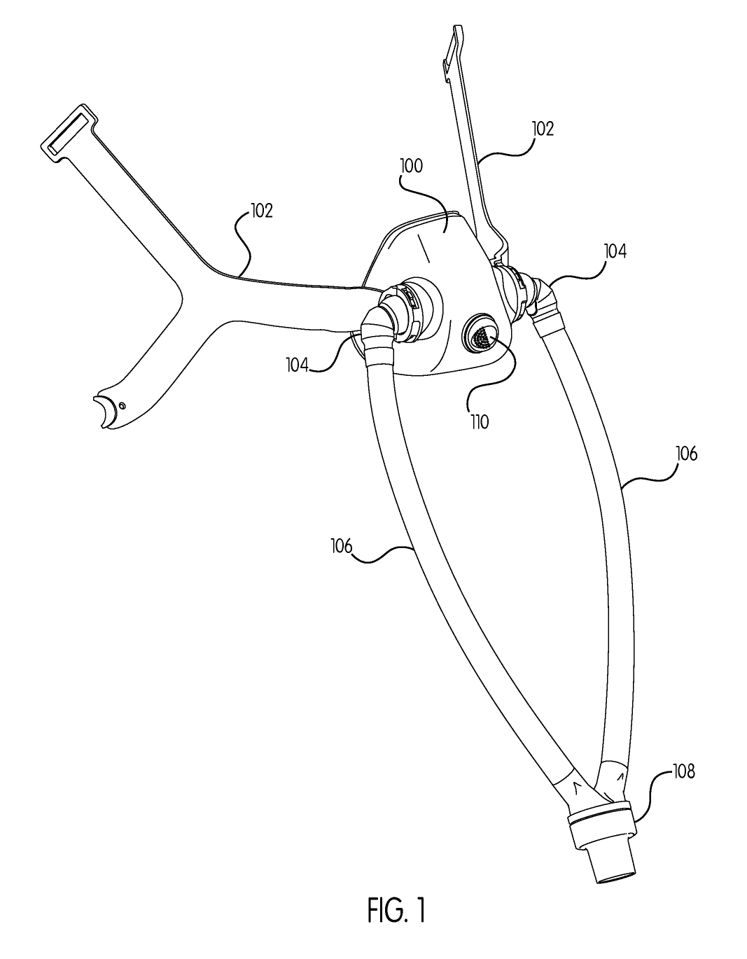

FIG. 1 shows a perspective view of the device. The device consists of mask member 100 which typically covers the user's nose, mouth or both. Mask member 100 is typically composed of a plastic material and will usually have a soft, sealing member attached to the edge where mask member 100 contacts the user's face (not shown). The sealing member is typically composed of soft silicon or silicon having a silicon gel filling. Exhaust port 110 is useful for removing exhaust air and moisture from the interior of mask member 100 during the exhalation phase of the breathing cycle.

Mask member 100 is held on the user's head via the headgear assembly consisting of the headgear members 102 and flexible straps, not shown. Headgear members 102 connect to mask member 100 at connection points each side of mask member 100. Air delivery ports 120 are preferably circular in shape and may be located anywhere on mask member 100.

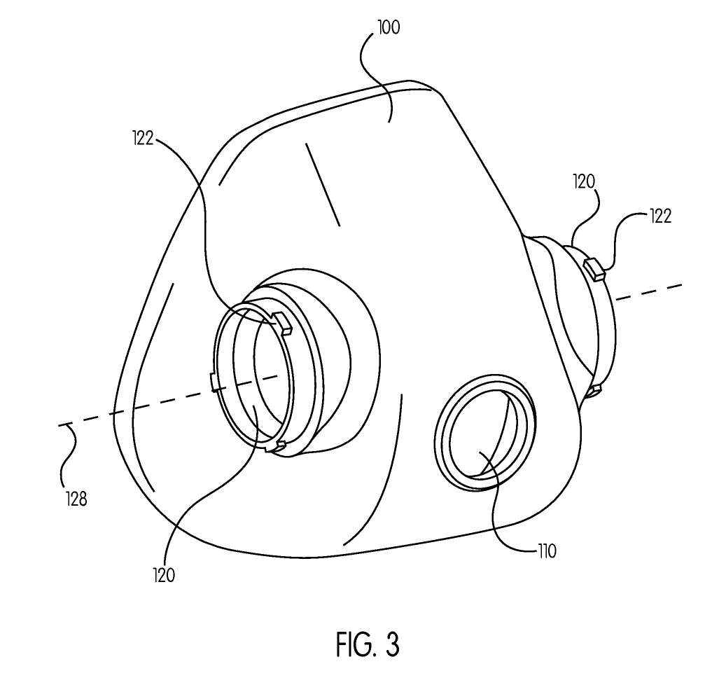

In a preferred embodiment of the invention, air delivery ports 120 are co-located at the connection points of the headgear members 102 and are held in place by connection portion 103 of headgear members 102. Details of air delivery ports 120 can be seen in FIG. 3 and include notches 122 that engage slots in the connection portions 103 of headgear members 102, which are also preferably circular in shape.

Headgear members 102 are preferably composed of a semi-rigid plastic or silicon, which retains its shape but which still may be flexed to assume the contour of the user's head. Headgear members 102 are preferably held on the user's head via flexible straps (not shown), preferably made of elastic, which attach to ends 129 of headgear members 102.

Headgear members 102 direct the forces pulling on mask member 100 horizontally and into the face and along and imaginary line through the ears of the user. This allows the force exerted by headgear members 102 to be transferred into mask member 100 and then managed by the user through angular adjustment of mask member 100, providing the desired amount of pressure at the preferred location.

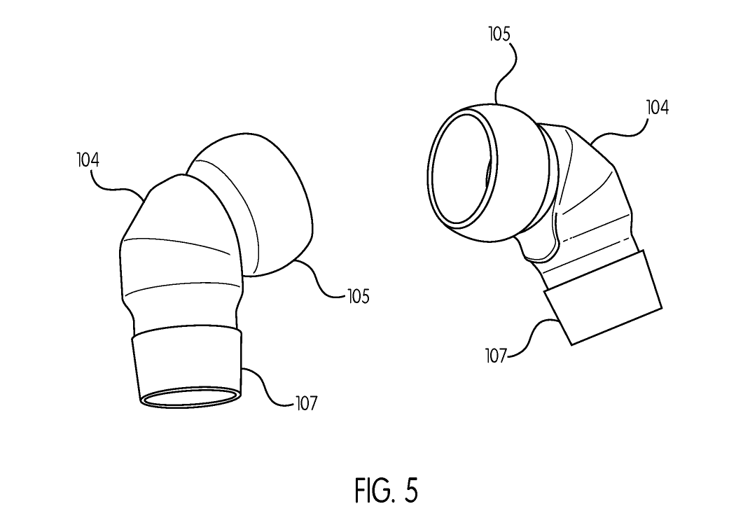

In the preferred embodiment wherein the air delivery ports 120 are co-located with the connection points of the headgear members 102, connector portions 103 of headgear members 102 also serve to hold hose connection members 104 to air delivery ports 120 on mask member 100. Hose connection members 104 are shown in detail in FIG. 5 and consist of rotational connection portion 105 and hose connection portion 107. Rotational connection portion 105 has a bulging, cylindrical shape with a maximum diameter slightly larger than connector portion 103 of headgear member 102. Hose connection member 104 is held in place by connector portion 103 of headgear member 102 when the slots in connector portion 103 engage tabs 122 of air delivery port 120, thereby allowing hose connection member 104 to rotate freely in any direction.

In various embodiments of the invention, the hose connection member 104 could either be free floating or be held in place via a friction adjustment or a positive lock. Hoses 106 are connected to hose connection portion 107 of hose connection member 104 and extend down at least far enough to clear the user's head and chin as hoses 106 rotate through the full range of motion.

One aspect of the novelty of the present device is now easily discerned. As the air delivery ports 120, the connector portions 103 of headgear members 102 and the rotational connection portions 105 of hose connection members 104 are all co-located and share axis of rotation 128, shown in FIG. 3, mask member 100 is able to rotate about axis 128, with respect to both the headgear members 102 and hose connection members 104, thereby providing the user with the ability to control tilt adjustment without the use of mechanisms typically found in the prior art, such as a forehead bar. This allows hoses 106 to rotate to any position, precluded only by the body of the user and prevents loading of mask member 100 reducing external forces, such as from the movement of hoses 106, that contribute to misalignment.

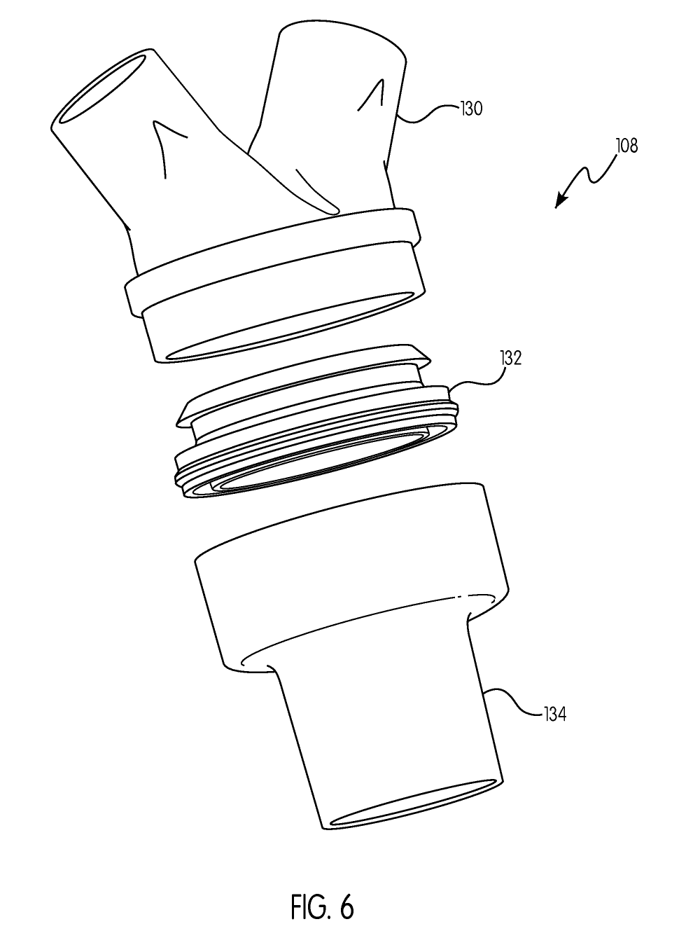

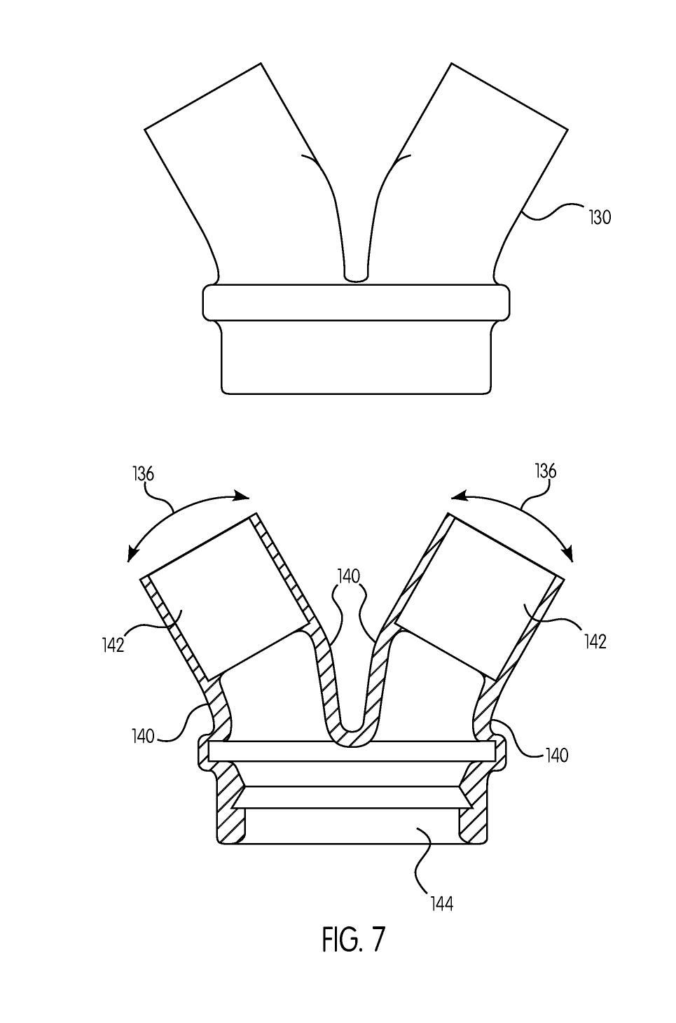

Breathing therapy machines typically have a singular hose connection, so it is necessary to provide Y-connector 108, shown in FIGS. 6 and 7. Y-connector 108 is shown in exploded view in FIG. 6 and consists of flexible member 130, coupler 132 and adapter portion 134. Flexible member 130 has connector portions 142 and 144, shown in FIG. 7, which engage the ends of hoses 106. Flexible membrane 140 is preferably composed of flexible silicon, and will allow the hoses to move through a range of motion in the directions shown by arrows 136 in FIG. 7, which can range from parallel up to an angle of approximately 50° without precluding airflow. Coupler portion 132, shown in FIG. 6, fits into recess 144 defined in flexible member 130, and connects to adapter portion 134. Preferably adapter portion 134 is allowed to rotate with respect to coupler portion 132, thereby allowing rotation of the hose from the CPAP machine as the patient moves during sleep. The neck of adapter portion 134 is sized to receive the single hose coming from the CPAP or Bi-PAP machine, and may or may not have some sort of fitting thereon to mate with the end of the breathing therapy machine hose. The design of Y-connector 108 is another aspect of the novelty of the device, allowing crimping of the flexible membrane 140, as the user moves during sleep, without obstructing airflow through hoses 106.

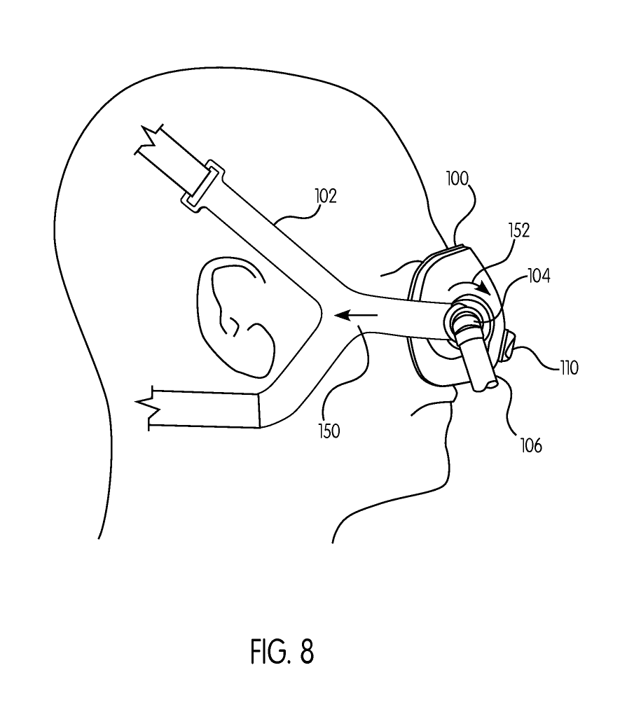

FIG. 8 shows the mask in place on a user. As can be seen, mask member 100 is held against the user's face via headgear straps, not shown, and headgear members 102, provide a line of force 150 which extends directly back through the user's ears. Rotational axis 128 allows the rotation of the mask member 100 and hoses 106 as shown by arrow 152.

Note that the point of rotation need not be located at the exact spot as shown in the drawings but may be placed at different locations on the mask to give a different feel and range of adjustability. In addition, it is not necessary that dual hoses be used. The device would work equally well with one hose or the other in which case one connection point would need to be sealed against air leakage. It has been found however that the device works best when used with two hoses, one on either side of the mask. As previously discussed, in alternative embodiments of the invention, the air delivery ports may not be co-located with the connection point of the headgear, but may be located elsewhere on the mask member. In such embodiments, the mask member is still free to rotate with respect to the headgear members, providing a similar advantages as the preferred embodiment.

The invention has been explained using exemplary embodiments and is not meant to be limited thereby. Variations which are still within the scope of the invention, as set forth in the following claims, are possible.

1. A mask for a breathing therapy machine comprising:
a mask member defining one or more air delivery ports;
a headgear member connected to each side of said mask member at a connection point;
wherein said mask member is able to rotate with respect to said headgear members about an axis passing through said connection points.

2. The mask of claim 1 further comprising:
one or more hose connection members, connected to a headgear member at said one or more air delivery ports, said hose connection members being able to rotate in any direction with respect to said mask member.
a hose connected at one end thereof to each of said hose connection members; and
a “Y” connector connected to the other end of each hose.

3. The mask of claim 2 wherein said “Y” connector is composed of a flexible material such as to allow bending of the portions connected to said hoses.

4. The mask of claim 1 wherein said headgear members hold said mask member against the face of a user with a force directed rearward along an line extending from the front of the user's head to the back, extending through the ears of said user.

5. The mask of claim 2 wherein said mask member, said headgear members and said hose connection members are all able to rotate about an axis extending through the center points of said air delivery ports.

6. The mask of claim 1 wherein said mask member comprises:
a rigid shell covering at least the nose of a user; and
a soft portion, connected to said rigid shell, said soft portion being disposed between said rigid shell and the face of said user.

7. The mask of claim 1 wherein said headgear members are connected to said mask via a circular connection member having notches defined therein which engage tabs defined on the circumference of said air delivery ports.

8. The mask of claim 7 wherein said headgear members are flexible such as to allow the ends of said headgear members opposite said circular connection members to bend around the head of a user.

9. The mask of claim 8 further comprising elastic strips engaging the ends of said headgear members for extending around the rear of the head of a user to hold said mask on said user's head.

10. The mask of claim 8 wherein the ends of said headgear members opposite said circular connection members are split into two legs to avoid contact with the ears of a user.

11. The mask of claim 7 wherein said hose connection members engage said circular connection members and are held in place when said circular connection members engage said air delivery ports.

12. The mask of claim 1 wherein said hose connection members comprise:
a bulged cylindrical portion disposed on one end thereof having a maximum diameter which is greater that the diameter of said air delivery ports;
a hose fitting on disposed on the opposite end thereof for connecting to a hose; and
a connecting tube therebetween.

13. The mask of claim 2 wherein said “Y” connector comprises:
a flexible material molded in a shape defining two outlets ports and one input port;
rigid circular members disposed on said two outlet ports for connecting to a hose;
a rigid circular member disposed on said inlet port for interfacing with an adaptive member; and
an adaptive member, connected directly or indirectly to said rigid circular member, said adaptive member sized to connect to a standard hose from said breathing therapy machine.

14. The mask of claim 13 further comprising a coupling member disposed between said adaptive member and said circular member, said coupling member allowing said adaptive member to rotate with respect to said circular member.

15. The mask of claim 13 wherein said flexible member allows said hoses to move through a range of motion from approximately vertical to at least 50 degrees from vertical.

16. The mask of claim 6 wherein said rigid shell is composed of silicon and further wherein said soft portion is composed of silicon or gel-filled silicon.

17. The mask of claim 1 further comprising:
one or more hose connection members, connected to a headgear member at said one or more air delivery ports, said hose connection members being able to rotate in any direction with respect to said mask member;
a hose connected at one end thereof to each of said hose connection members; and
a manifold, connecting the other opposite end of said one of more hoses.

18. The mask of claim 18 further comprising elastic strips engaging the ends of said headgear members for extending around the rear of the head of a user to hold said mask on said user's head.

19. A mask for a breathing therapy machine comprising:
a mask member defining dual co-axial air delivery ports;
a headgear member connected to said mask member at each of said air delivery ports, said mask member and said head gear member being able to rotate with respect to each other about an axis passing through said co-axial air delivery ports; and
one or more hose connection members, connect to a headgear member at said air delivery ports, said hose connection members being able to rotate in any direction with respect to said mask member
